(19) United States
(12) Patent Application Publication
(10) Pub. No.: US 2011/0153001 A1
(43) Pub. Date: Jun. 23, 2011

(54) BIODEGRADABLE STENT

(75) Inventors: SHIH-JUNG LIU, TAIPEI CITY (TW); FU-JYUN JIANG, TAIPEI COUNTY (TW)

(73) Assignee: CHANG GUNG UNIVERSITY, TAOYUAN COUNTY (TW)

(21) Appl. No.: 12/641,949

(22) Filed: Dec. 18, 2009

(51) Int. Cl. A61F 2/82

(52) U.S. Cl. 623/1.16

(57) ABSTRACT

A biodegradable stent includes flexible connection units comprising extensions and a base perpendicular thereto. The base includes ring members coupled by links. The extension includes a first end, a ring element at a second end and a hollow double-convex shaped intermediate section. The width of the intermediate section is greater than an inner diameter of each of the ring members and each of the ring elements. The ring elements of the extensions of a first connection unit are inserted through the ring members of the extensions of a second connection unit to assemble the first and second connection units. The connection units are connected and securely joined to form a contracted tubular stent. The base of the second connection unit is superimposed with the second end of the first connection unit. In such a manner the stent is expanded.

### BACKGROUND OF THE INVENTION

1. Field of Invention

The invention relates to stents, and more particularly, to a biodegradable stent with improved characteristics.

2. Description of Related Art

Heart related diseases (e.g., coronary heart diseases) are common among people in countries throughout the world. It is known that deposition of fat resulted from cholesterol in the arteries can cause atherosclerosis which hardens or narrows the arteries. This is so-called sclerosis. Another disease is thrombosis which results from the formation of blood clots (i.e., thrombus) inside a blood vessel and can obstruct the flow of blood.

One effective method of treating a heart related disease is the use of stent. In detail, a stent is a man-made tube inserted into a natural conduit (e.g., blood vessel) in the body with the aid of a catheter. Next, a cylindrical web structure around the stent is removed. The stent thus automatically expands due to its elastic nature. As a result, the blood vessel is held open to allow access for surgery.

Alternatively, a stent inserted into a blood vessel with the aid of a catheter which is in turn mounted with an angioplasty balloon. Next, a cylindrical web structure is mounted around the stent. Then, the angioplasty balloon inflates automatically to expand the stent. As a result, the blood vessel is held open by the stent to allow access for surgery. Next, the angioplasty balloon is deflated so that the web structure can be removed thereafter.

For holding a blood vessel open for a relatively long period of time, stents are typically made of metal with elasticity. However, it is impossible for a human body to degrade metal. Disadvantageously, the metallic stents remained in the blood vessels may cause abnormal blood coagulation such as thrombosis.

For eliminating drawbacks associated with metallic stents, biodegradable stents have been devised. In detail, these stents are made of a biodegradable material such as polylactic acid (PLA), polyglycolic acid (PGA), polycaprolactone (PCL), copolymer thereof, or derivative thereof. These biodegradable materials are typically subject to a softening process by means of a solvent such as acetone, methyl dichlorosilane, chloroform. However, such produced biodegradable stents may have the toxic components contained in the remained solvent. Further, the toxic components may degrade and remain in the human body. This can cause diseases and harm our body.

In addition, typical metallic or biodegradable stents are web or spiral structures. However, no permanent fastening mechanism is provided by the typical stents. Hence, the structural strength of the typical stents may decrease gradually due to the contraction of walls of the blood vessels. Thus, a need for improvement exists.

### SUMMARY OF THE INVENTION

It is therefore one object of the invention to provide a biodegradable stent.

To achieve the above and other objects, the invention provides a biodegradable stent comprising a plurality of flexible connection units formed by a biodegradable material and comprising a plurality of extensions and a base perpendicular thereto. Wherein, the base comprises a plurality of closed loop members and a plurality of links each for coupling two of the closed loop members together, and the extension comprises a first end, a second end, a closed loop element extending out of the second end, and an intermediate section between the first and second ends, the intermediate section having a through hole; wherein the intermediate section is substantially shaped as a double-convex, and the width of the intermediate section of the extension is greater than an inner diameter of each of the closed loop members and the closed loop elements; wherein the closed loop elements of the extensions of a first connection unit are inserted through the closed loop members of the extensions of a second connection unit to be disposed below the second ends of the extensions of the second connection unit with the closed loop members of the base of the second connection unit looped around the first ends of the extensions of the first connection unit to assemble the first and second connection units; wherein the base of the second connection unit is adapted to superimpose with the intermediate section of the immediately previous first connection unit by pulling until all of the connection units are connected together; wherein the extension of a last one of the connection units is bent to put on the base of the first connection unit for engagement; wherein the engagement is heated to form the contracted tubular stent; and wherein the base of the second connection unit is adapted to superimpose with the second end of the first connection unit by pulling until all of the connection units are pulled to form the expanded tubular stent.

In one aspect of the invention the intermediate section has a lengthwise through hole.

In another aspect of the invention the biodegradable material is selected from one of the groups consisting of polylactic acid (PLA), polyglycolic acid (PGA), polycaprolactone (PCL), polydioxanone (PDX), polyglactin, PCL-PGA copolymer, and polyglyconate.

In a yet another aspect of the invention the heating of the engagement is done by micro injection molding.

In a further aspect of the invention each of the closed loop members and the closed loop elements is a ring.

In a yet further aspect of the invention each of the closed loop members and the closed loop elements has a rectangular hole.

By utilizing the invention, the following advantages can be obtained. Toxic solvent is not involved in the manufacturing processes of the biodegradable stents. Instead, micro injection molding is involved. The stent stayed in the human body causes no harm because it will not release toxic components. The stent can easily expand from its contracted state in human body insertion process.

The above and other objects, features and advantages of the invention will become apparent from the following detailed description taken with the accompanying drawings.

### BRIEF DESCRIPTION OF THE DRAWINGS

FIG. 1 is a flowchart depicting a process for manufacturing the biodegradable stent according to a preferred embodiment of the invention;

FIG. 2 is a flowchart depicting a process for manufacturing the connection unit of FIG. 1;

FIG. 3 is a perspective view of a first configuration of the connection unit;

FIG. 4 is a perspective view of a second configuration of the connection unit;

FIG. 5 is a perspective view of a third configuration of the connection unit;

FIG. 6 is a perspective view of one connection unit of the first configuration to be assembled with another connection unit having the same configuration;

FIG. 7 is a perspective view of the connection units of FIG. 6 being assembled in a first fashion;

FIG. 8 is a view similar to FIG. 7 where the connection units of FIG. 7 are assembled in a second fashion;

FIG. 9 is a view similar to FIG. 7 where the connection units of FIG. 7 are assembled in a third fashion;

FIG. 10 is a perspective view of four connection units of the first configuration assembled;

FIG. 11 is a perspective view of the connection units of FIG. 10 to be secured onto a heating plate;

FIG. 12 is a perspective view of the connection units secured onto the heating plate;

FIG. 13 is a perspective view of the connection units of FIG. 12;

FIG. 14 is a side elevation of the stent of FIG. 13 being further contracted;

FIG. 15 is a side elevation of the stent of FIG. 13 being further expanded; and

FIGS. 16A to 16D are perspective views of fourth, fifth, sixth, and seventh configurations of the connection unit respectively.

### DETAILED DESCRIPTION OF THE INVENTION

A biodegradable stent of the invention is adapted to insert into a natural passage or conduit (e.g., blood vessel, trachea, urethra, and intestine) in the body to prevent or counteract a disease-induced, localized flow constriction.

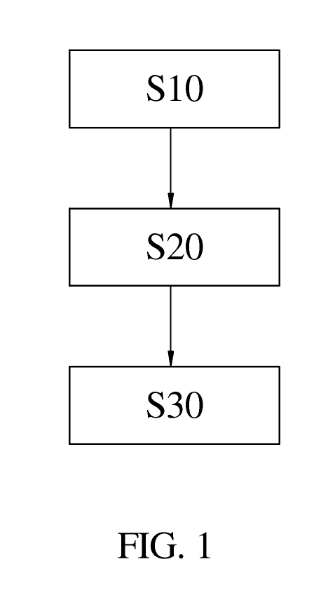

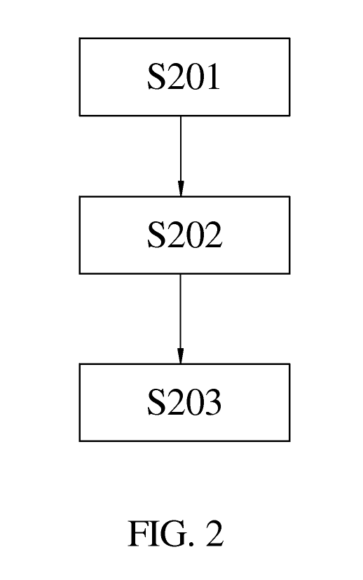

Referring to FIGS. 1 and 2, a process for manufacturing the biodegradable stent according to a preferred embodiment of the invention is illustrated. As shown, a biodegradable material is selected (step S10). Note that the biodegradable material can be, for example, PCL, PLA, PGA, polydioxanone (PDX), polyglactin, PCL-PGA copolymer, or polyglyconate. Next, a connection unit is produced (step S20). In the production of connection unit (see FIG. 2), a predetermined connection unit mold is selected (step S201), a programmable precision carving machine is employed to carve a core to form the connection unit mold (step S202), and the selected biodegradable material (e.g., PCL) is fed into and heated in the connection unit mold to be subject to micro injection molding by means of a micro injection molding machine (step S203). Finally, a biodegradable stent is produced by assembling a plurality of connection units (step S30).

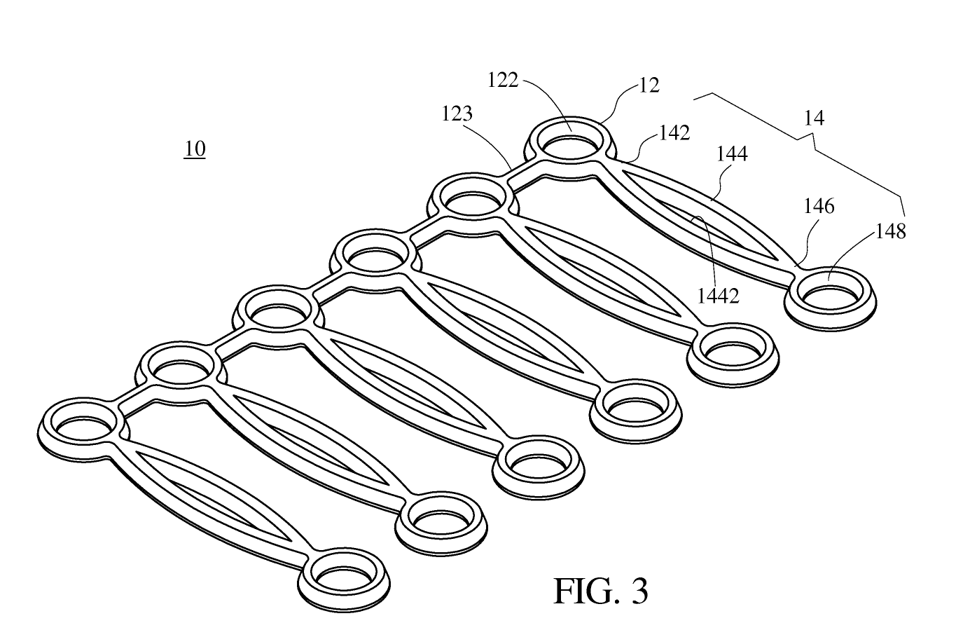

Referring to FIG. 3, a first configuration of a connection unit 10 comprises a plurality of extensions 14 and a base 12 formed integrally. The connection unit 10 is flexible in nature. The elongated base 12 comprises a plurality of rings 122 coupled together by links 123. The elongated extension 14 is perpendicular to the base 12 and comprises a first end 142 formed with the ring 122, a second end 146, a ring 148 extending out of the second end 146, and an intermediate section 144 between the first and second ends 142, 146. The intermediate section 144 has a central through hole 1442 shaped as a double-convex. The intermediate section 144 and the first and second ends 142, 146 together are shaped as a double-convex. The width of the intermediate section 144 is greater than an inner diameter of each of the rings 122, 148.

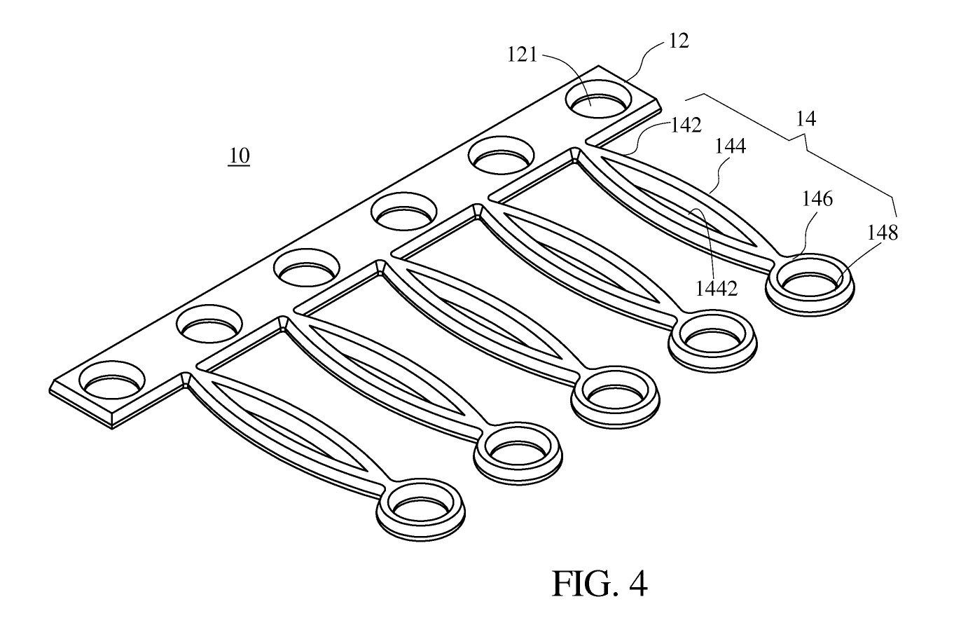

Referring to FIG. 4, a second configuration of the connection unit 10 is shown. The characteristic of the second configuration is that the base 12 is shaped as a rectangle with a plurality of equally spaced holes 121 formed therein.

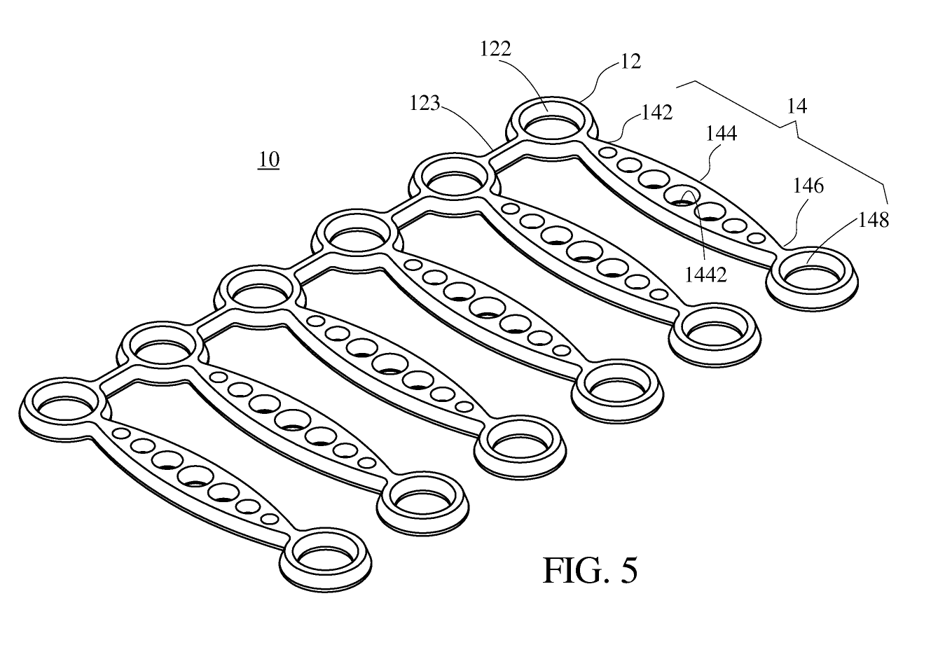

Referring to FIG. 5, a third configuration of the connection unit 10 is shown. The characteristic of the third configuration is that the double-convex shaped central through hole 142 is replaced with a plurality of lengthwise through holes 1442 of different diameters in which the middle through hole 1442 has the largest diameter and either end through hole 1442 has the smallest diameter.

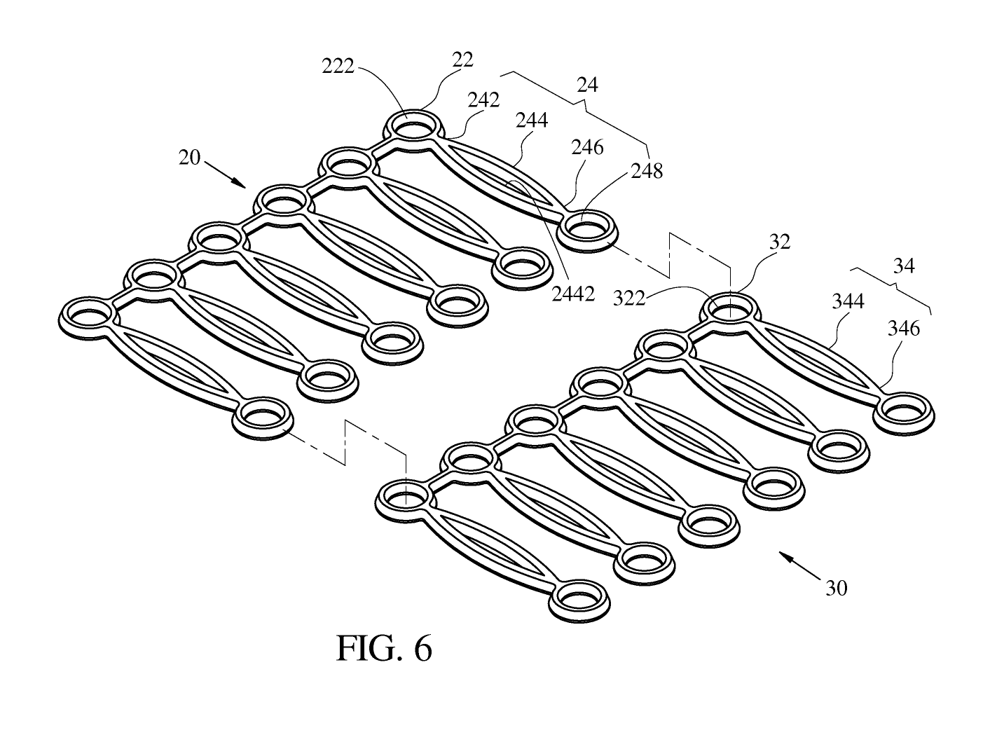

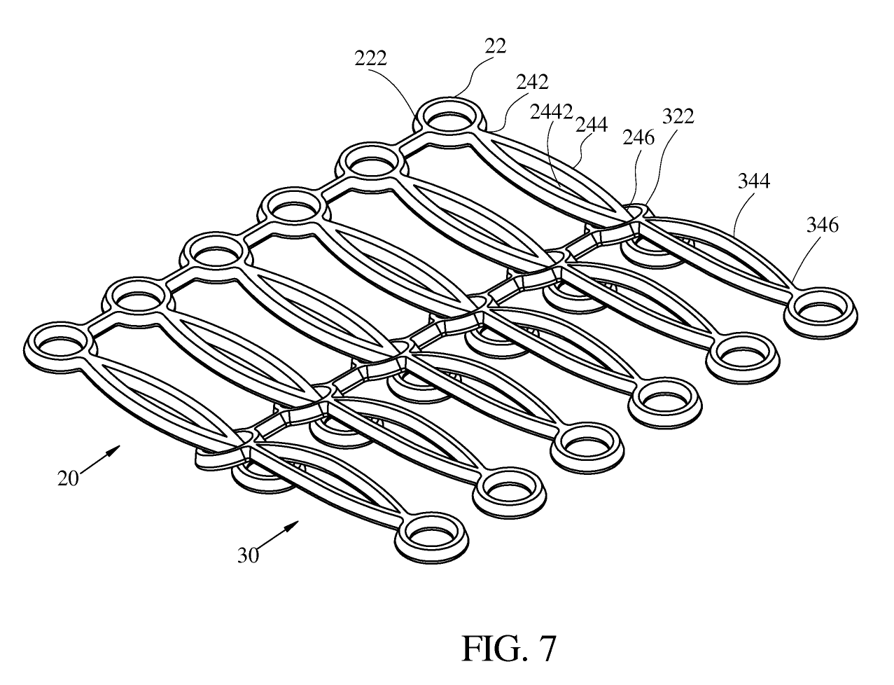

Referring to FIGS. 6, 7, and 8, an assembly of connection units 20, 30 of the first configuration is shown. Rings 248 of extensions 24 of a first connection unit 20 are inserted through the rings 322 of extensions 34 of a second connection unit 30 to be disposed below second ends 346 of the extensions 34 of the second connection unit 30 with rings 322 of a base 32 of the second connection unit 30 looped around first ends 242 of the extensions 24 of the first connection unit 20. As a result, the first and second connection units 20, 30 are assembled.

The assembly is reliable (i.e., being not susceptible of disengagement) because as stated above each extension has a double-convex shape and the width of the intermediate section of each extension is greater than an inner diameter of each ring. Moreover, the base 32 of the second connection unit 30 may slide between the first end 242 and the second end 246. This is because, as stated above, the connection units are flexible in nature.

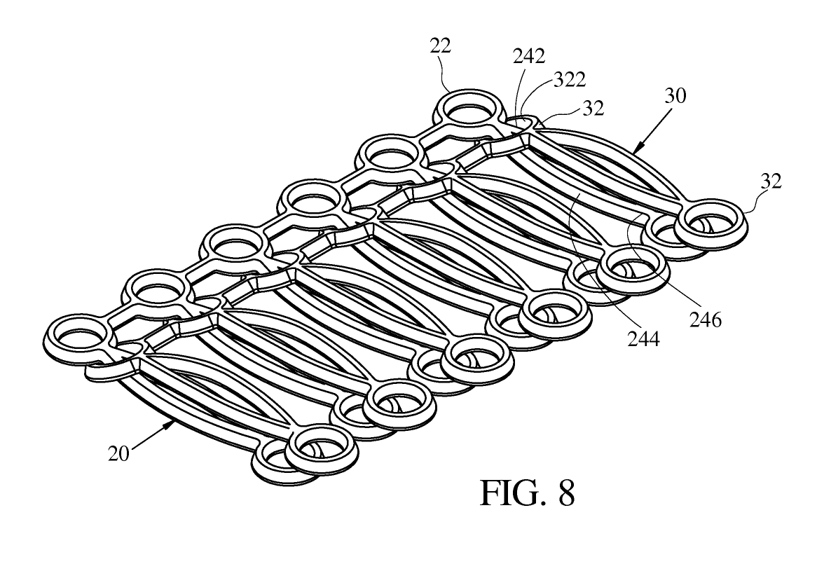

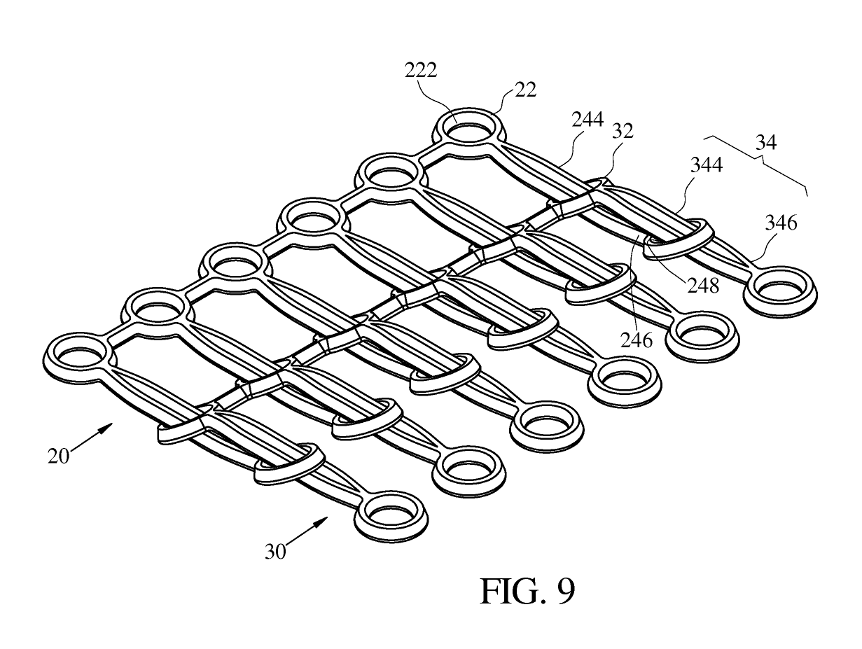

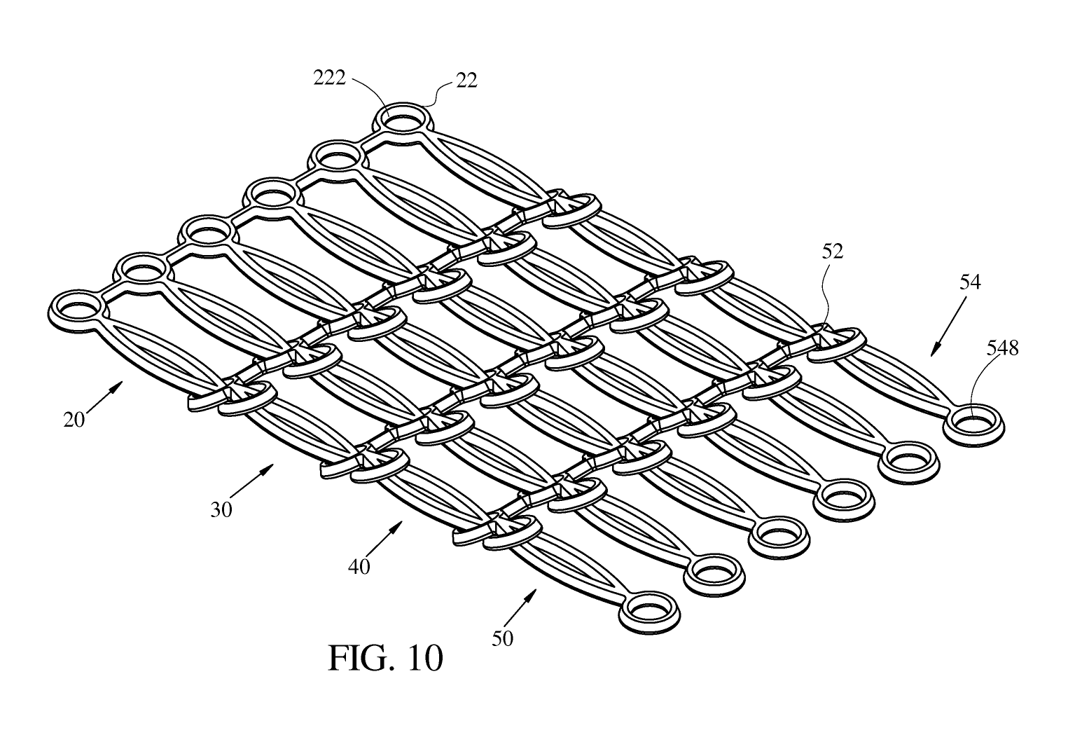

Referring to FIGS. 9 and 10 in conjunction with FIG. 8, a medical employee may insert the rings of the extensions of the second connection unit 30 through the rings of the extensions of the first connection unit 20. Next, the medical employee may pull the rings of the extensions of the second connection unit 30 until together with the first connection unit 20 a shape is formed (see FIGS. 8 and 9). Thereafter, the medical employee may pull the second connection unit 30 until a maximum length of the assembled first and second connection units 20, 30 is obtained (see the left part of FIG. 10).

Thereafter, third and fourth connection units 40, 50 are assembled with the second connection unit 30 in a manner as described in the above paragraphs. Finally, the first, second, third, and fourth connection units 20, 30, 40, and 50 are assembled (see FIG. 10).

The number of connection units to be assembled depends on the bore of a natural conduit (e.g., blood vessel) of the body. That is, the larger of the bore of, for example, a blood vessel the greater of the number of the connection units to be assembled and vice versa so that the produced stent can be inserted into the blood vessel. Four connection units 20, 30, 40, and 50 are employed in the embodiment.

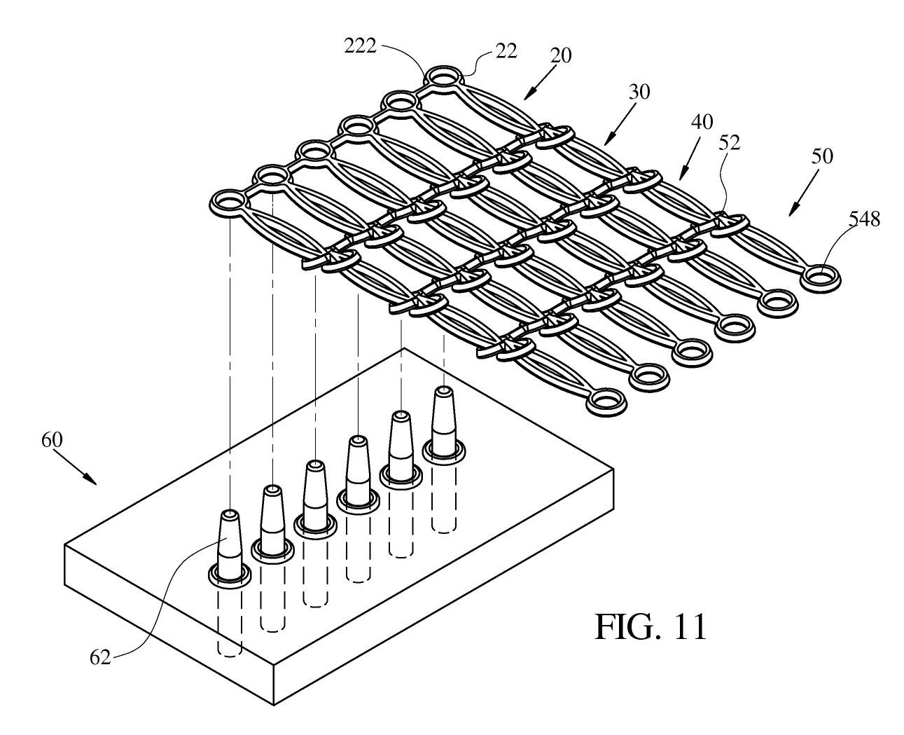

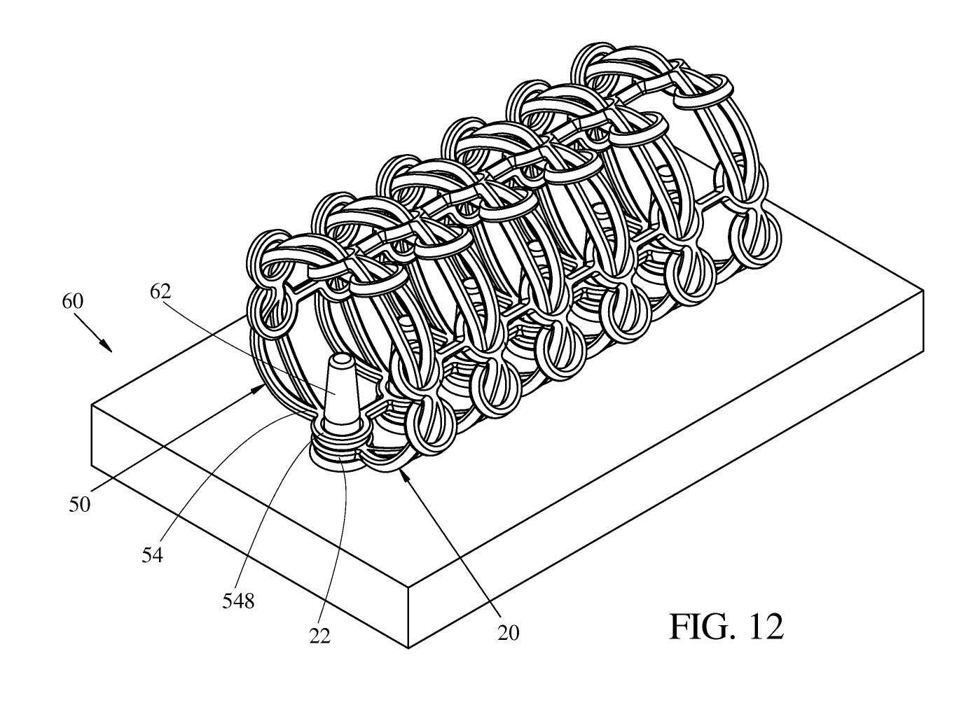

Referring to FIGS. 11, 12 and 13, a parallelepiped heating plate 60 is provided. The heating plate 60 comprises a plurality of heat conductive cylindrical members 62 projecting upward along a lengthwise central line. The heat conductive cylindrical members 62 are made of a good heat conductive material such as iron, aluminum, or copper. The heat conductive cylindrical members 62 have a tapered end and a main portion having an outer diameter substantially the same as the bore of the ring 222 or 548. Hence, the heat conductive cylindrical members 62 are adapted to insert through the rings 222 of the base 22 of the first connection unit 20 which is assembled with the second, third, and fourth connection units 30, 40, and 50. Further, the rings 548 are put on the heat conductive cylindrical members 62 after bending the assembled second, third, and fourth connection units 30, 40, and 50. As a result, the assembled second, third, and fourth connection units 30, 40, and 50 are disposed on the heating plate 60 (see FIG. 12).

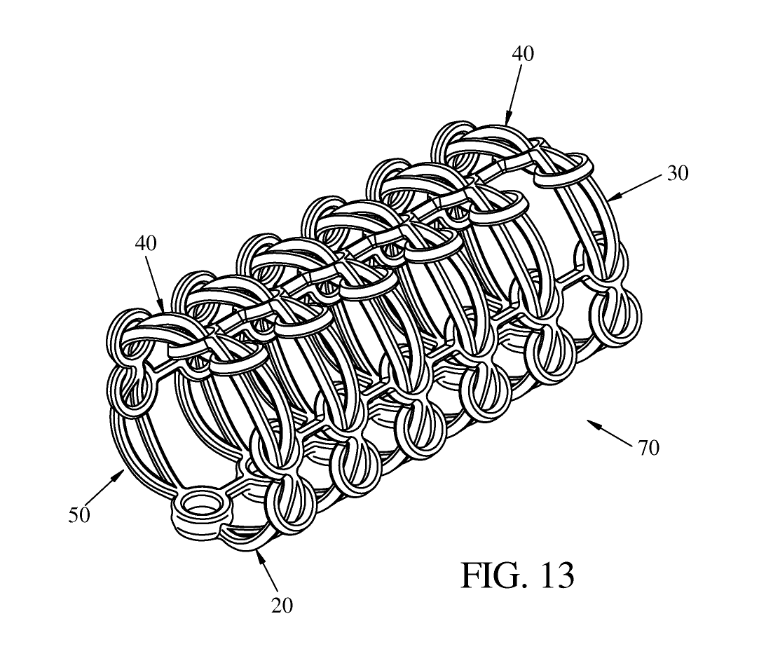

Thereafter, a heating device (not shown) is employed to heat the heating plate 60 and the heat conductive cylindrical members 62. As such, the base 22 of the first connection unit 20 and the rings 548 of the extensions 54 of the fourth connection unit 50 are joined due to red heat. As a result, a tubular biodegradable stent 70 is produced (see FIG. 13).

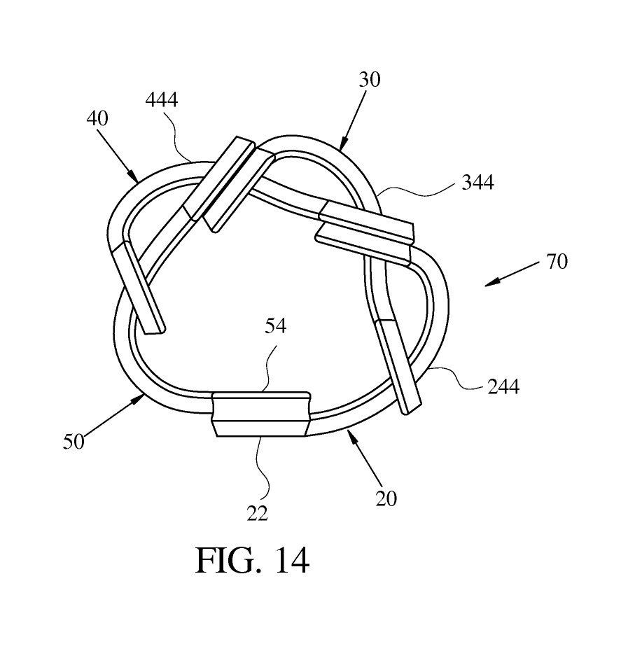

Referring to FIG. 14 in conjunction with FIG. 12, a medical employee may pull the base 32 of the second connection unit 30 to superimpose with the intermediate section 244 of the first connection unit 20, pull the base 42 of the third connection unit 40 to superimpose with the intermediate section 344 of the second connection unit 30, and pull the base 52 of the fourth connection unit 50 to superimpose with the intermediate section 444 of the third connection unit 40 prior to bending the extension 54 of the fourth connection unit 50 to put on the base 22 of the first connection unit 20. As a result, the stent 70 is contracted.

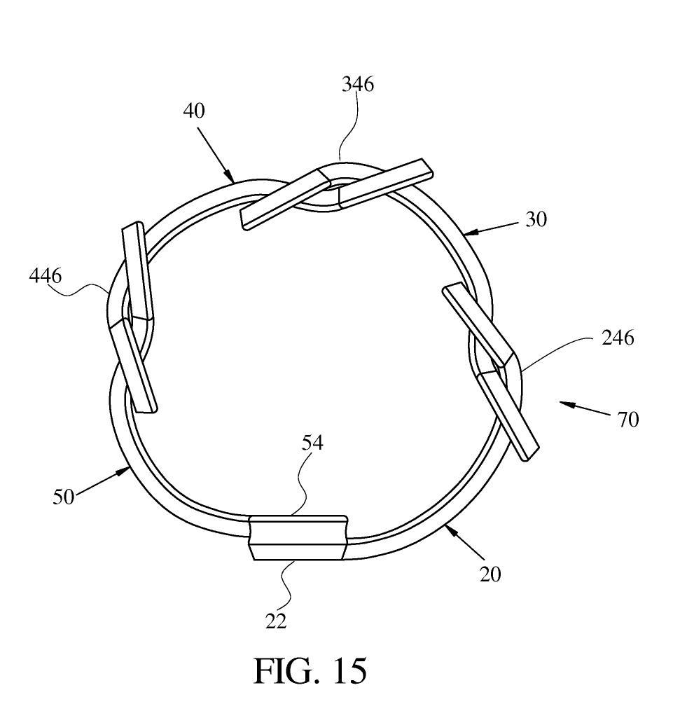

Referring to FIG. 15 in conjunction with FIG. 12, a medical employee may pull the base 32 of the second connection unit 30 to superimpose with the second end 246 of the first connection unit 20, pull the base 42 of the third connection unit 40 to superimpose with the second end 346 of the second connection unit 30, and pull the base 52 of the fourth connection unit 50 to superimpose with the second end 446 of the third connection unit 40 prior to bending the extension 54 of the fourth connection unit 50 to put on the base 22 of the first connection unit 20. As a result, the stent unit 70 is expanded.

A medical employee may choose to use the expanded stent or the contracted stent. In one exemplary example, a medical employee inserts the contracted biodegradable stent into a natural conduit (e.g., blood vessel) with the aid of a catheter which is in turn mounted with an angioplasty balloon. Next, a cylindrical web structure is mounted around the stent. Next, the angioplasty balloon inflates automatically to expand the stent. And in turn, the base 32 of the second connection unit 30 slides to the second end 246 of the first connection unit 20, the base 42 of the third connection unit 40 slides to the second end 346 of the second connection unit 30, and the base 52 of the fourth connection unit 50 slides to the second end 446 of the third connection unit 40. As a result, the blood vessel is held open by the expanded stent to allow access for surgery.

It is noted that the stent is held in place because, as stated above, each extension has a double-convex shape and the width of the intermediate section of each extension is greater than an inner diameter of each ring. Moreover, the base of a connection unit may slide between the first end and the second end of another connected connection unit.

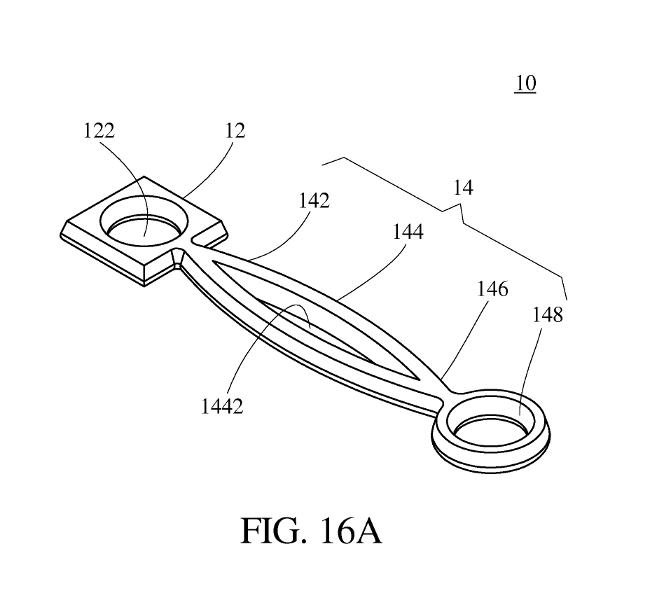

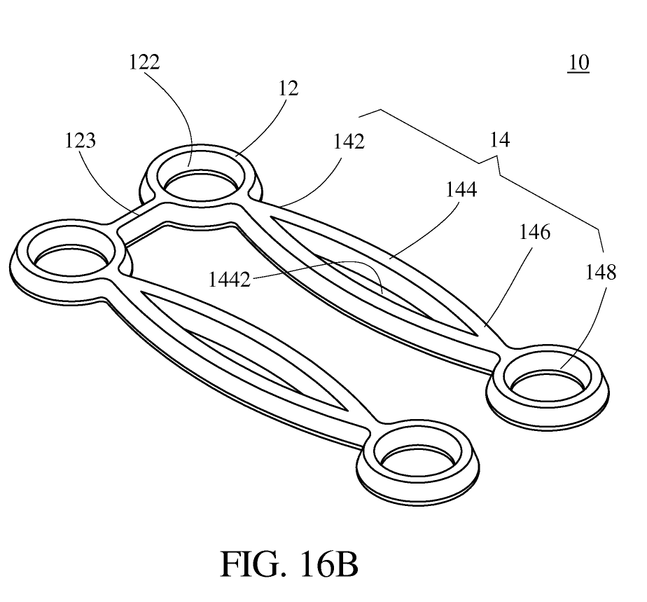

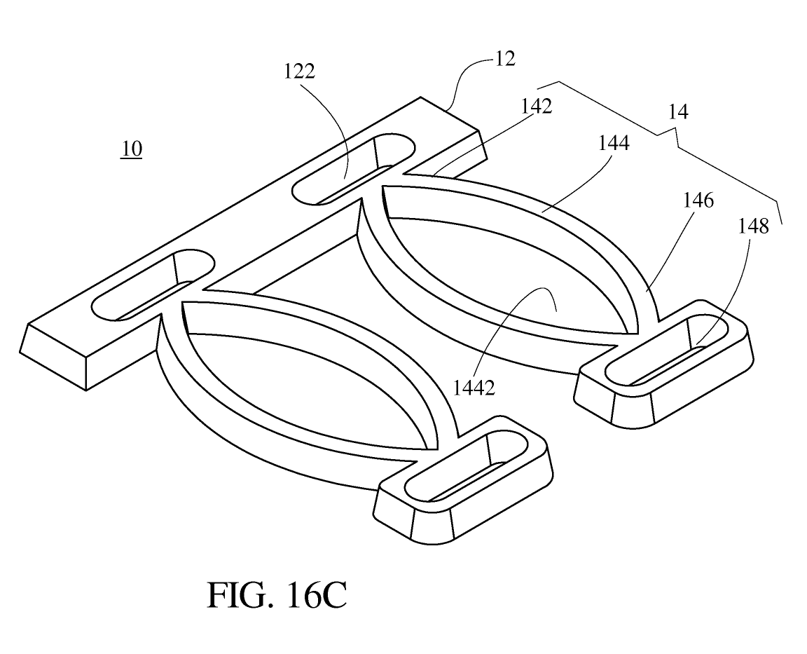

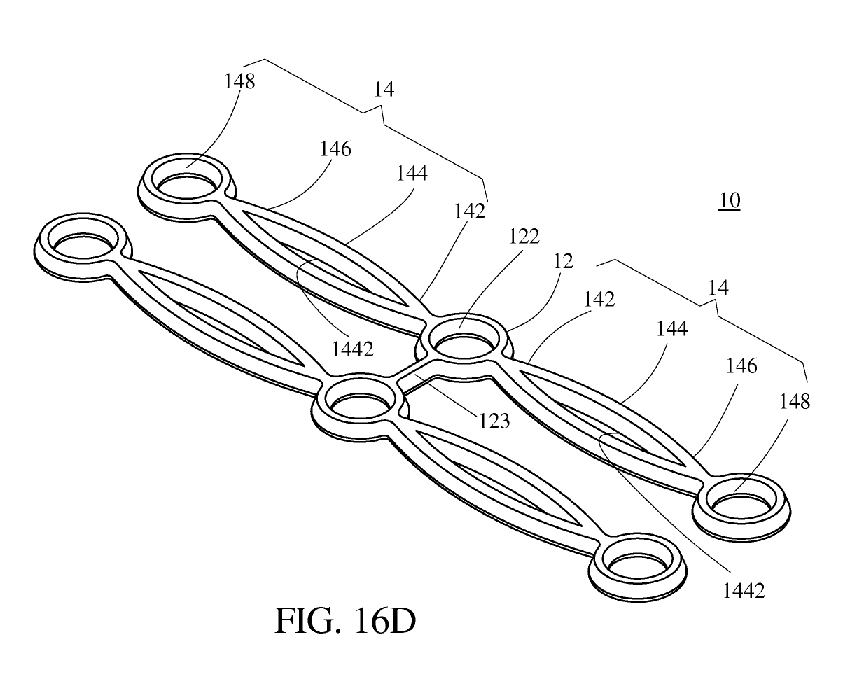

The number of the extension(s) of the connection unit depends on the bore of a natural conduit. For example, referring to FIG. 16A, a fourth configurations of the connection unit 10 is shown. The connection unit 10 comprises a rectangular base 12 having a hole 122 and an integral extension 14 perpendicular to the base 12, the extension 14 including a first end 142 formed with the base 12, a second end 146 distal the base 12, a ring 148 extending out of the second end 146, and a double-convex shaped intermediate section 144 between the first and second ends 142 and 146, the intermediate section 144 having a central through hole 1442 shaped as a double-convex.

Referring to FIG. 16B, a fifth configuration of the connection unit 10 is shown. The connection unit 10 comprises a base 12 having two rings 122 interconnected by a link 123 and two integral parallel extensions 14 perpendicular to the base 12, the extension 14 including a first end 142 formed with either ring 122 of the base 12, a second end 146 distal the base 12, a ring 148 extending out of the second end 146, and a double-convex shaped intermediate section 144 between the first and second ends 142 and 146, the intermediate section 144 having a central through hole 1442 shaped as a double-convex.

Referring to FIG. 16C, a sixth configuration of the connection unit 10 is shown. The connection unit 10 comprises a rectangular base 12 having two holes 122 and two integral parallel extensions 14 perpendicular to the base 12, the extension 14 including a first end 142 formed with the base 12, a second end 146 distal the base 12, a closed loop 148 extending out of the second end 146, and a double-convex shaped intermediate section 144 between the first and second ends 142 and 146, the intermediate section 144 having a central through hole 1442 shaped as a double-convex.

Referring to FIG. 16D, a seventh configuration of the connection unit 10 is shown. The connection unit 10 comprises a base 12 having two rings 122 interconnected by a link 123 and two opposite sets of two integral parallel extensions 14 perpendicular to the base 12, the extension 14 including a first end 142 formed with either ring 122 of the base 12, a second end 146 distal the base 12, a ring 148 extending out of the second end 146, and a double-convex shaped intermediate section 144 between the first and second ends 142 and 146, the intermediate section 144 having a central through hole 1442 shaped as a double-convex.

The biodegradable stent of the invention has the following advantages. A plurality of flexible connection units is assembled as a contracted stent which is in turn pulled to form an expanded stent. The stent is reliable (i.e., being not susceptible of disengagement) because each extension of the connection unit has a double-convex shape and the width of intermediate section of each extension is greater than an inner diameter of each ring. Moreover, the base of a connection unit may slide between first and second ends of another connected connection unit. The material of manufacturing the stent is biodegradable. The selected biodegradable material is fed into and heated in a mold to be subject to micro injection molding. Finally, a biodegradable stent is produced. Advantageously, toxic solvent is not involved in the manufacturing processes of the biodegradable stents. The stent stayed in the human body causes no harm because its material is biodegradable without toxic components. This is a great advancement as compared with the prior art since the conventional metallic stent stayed in the body releases toxic components from the remained solvent to cause diseases.

While the invention herein disclosed has been described by means of specific embodiments, numerous modifications and variations could be made thereto by those skilled in the art without departing from the scope and spirit of the invention set forth in the claims.

What is claimed is:

1. A biodegradable stent comprising:
a plurality of connection units formed by a biodegradable material and comprising a plurality of extensions and a base perpendicular thereto;
wherein the base comprises a plurality of closed loop members and a plurality of links each for coupling two of the closed loop members together, and each of the extensions comprises a first end, a second end, a closed loop element extending out of the second end, and an intermediate section between the first and second ends;
wherein the intermediate section is substantially shaped as a double-convex, and a width of the intermediate section of the extension is greater than an inner diameter of each of the closed loop members and the closed loop elements;
wherein the closed loop elements of the extensions of a first connection unit are inserted through the closed loop members of the extensions of a second connection unit to dispose below the second ends of the extensions of the second connection unit with the closed loop members of the base of the second connection unit looped around the first ends of the extensions of the first connection unit to assemble the first and second connection units;
wherein the base of the second connection unit is adapted to superimpose with the intermediate section of an immediately previous first connection unit by pulling until all of the connection units are connected together;
wherein the extension of a last one of the connection units is bent to put on the base of the first connection unit for engagement;
wherein the engagement is heated to form a contracted tubular stent; and
wherein the base of the second connection unit is adapted to superimpose with the second end of the first connection unit by pulling until all of the connection units are pulled to form an expanded tubular stent.

2. The biodegradable stent of claim 1, wherein the intermediate section has a lengthwise through hole.

3. The biodegradable stent of claim 1, wherein a material of the connection units is selected from the group consisting of one of polylactic acid (PLA), polyglycolic acid (PGA), polycaprolactone (PCL), polydioxanone (PDX), polyglactin, PCL-PGA copolymer, and polyglyconate.

4. The biodegradable stent of claim 1, wherein the connection units are formed by micro injection molding.

5. The biodegradable stent of claim 1, wherein the first end is formed with the closed loop member.

6. The biodegradable stent of claim 1, wherein each of the closed loop members and the closed loop elements is a ring.

7. The biodegradable stent of claim 1, wherein each of the closed loop members and the closed loop elements has a rectangular hole.
